(19) United States
(12) Patent Application Publication
(10) Pub. No.: US 2024/0060176 A1
(43) Pub. Date: Feb. 22, 2024

(54) RAW MATERIAL FOR CHEMICAL DEPOSITION CONTAINING ORGANORUTHENIUM COMPOUND, AND CHEMICAL DEPOSITION METHOD FOR RUTHENIUM THIN FILM OR RUTHENIUM COMPOUND THIN FILM

(71) Applicant: TANAKA KIKINZOKU KOGYO K.K., Tokyo (JP)

(72) Inventors: Kazuharu SUZUKI, Tsukuba-shi (JP); Yuki MORI, Tsukuba-shi (JP); Subhabrata DAS, Tsukuba-shi (JP); Hirofumi NAKAGAWA, Tsukuba-shi (JP); Shunichi NABEYA, Tsukuba-shi (JP)

(73) Assignee: TANAKA KIKINZOKU KOGYO K.K., Tokyo (JP)

(21) Appl. No.: 18/281,267

(22) PCT Filed: Feb. 22, 2022
(86) PCT No.: PCT/JP2022/007082

§ 371 Date: Sep. 8, 2023

(30) Foreign Application Priority Data
Mar. 10, 2021 (JP) 2021-038084

(51) Int. Cl. C23C 16/18; C23C 16/448

(52) U.S. Cl. CPC C23C 16/18; C23C 16/4486

(57) ABSTRACT

The present invention is drawn to a raw material for chemical deposition for producing a ruthenium thin film or a ruthenium compound thin film by a chemical deposition method, containing an organoruthenium compound represented by the following formula 1, and further containing β-diketone that is the same as a ligand of the organoruthenium compound. The raw material for chemical deposition of the present invention is inhibited in discoloration/precipitation even when heated at a high temperature, and enables to form a stable ruthenium thin film or ruthenium compound thin film.

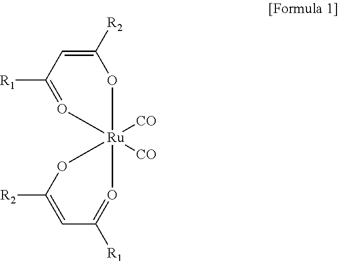

wherein substituents R₁ and R₂ are each hydrogen, or a linear or branched alkyl group.

### CROSS-REFERENCE TO RELATED APPLICATIONS

The present application claims priority under 35 U.S.C. § 371 to International Patent Application No. PCT/JP2022/007082, filed on Feb. 22, 2022, which claims priority to and the benefit of Japanese Patent Application No. 2021-038084, filed on Mar. 10, 2021. The contents of these applications are hereby incorporated by reference in their entireties.

### BACKGROUND OF THE INVENTION

### Field of the Invention

The present invention relates to a raw material for chemical deposition containing an organoruthenium compound as a principal component for producing a ruthenium thin film or a ruthenium compound thin film by a chemical deposition method (chemical vapor deposition method (CVD method)), or an atomic layer deposition method (ALD method). The present invention also relates to a method for producing a ruthenium thin film or a ruthenium compound thin film by a chemical deposition method.

### Description of the Related Art

Ruthenium (Ru) has a low resistance and thermal/chemical stability, and hence is suitable as a wiring/electrode material of various semiconductor devices. In particular, a ruthenium thin film is used as a seed layer or a liner layer in a wiring structure of a semiconductor device, a gate electrode in a transistor, a capacitor electrode in a memory, and the like. As a method for producing a ruthenium thin film applied to these, a chemical deposition method such as a CVD method (chemical vapor deposition method), or an ALD method (atomic layer deposition method) is applied.

As a raw material for chemical deposition (precursor) used in the chemical deposition method, a large number of organoruthenium compounds have been conventionally reported. The present applicant also has developed and disclosed a large number of organoruthenium compounds suitable as the raw material for chemical deposition. Among these, an organoruthenium compound to be put to practical use owing to vaporization properties/film formation properties thereof includes a raw material for chemical deposition containing an organoruthenium compound of the formula 2 that is represented by dicarbonylbis(2-methyl-4-hexen-3-one-5-oxide)ruthenium (II) of the following formula 1 (hereinafter referred to as the “Ru complex 1”) (see Patent Documents 1 and 2).

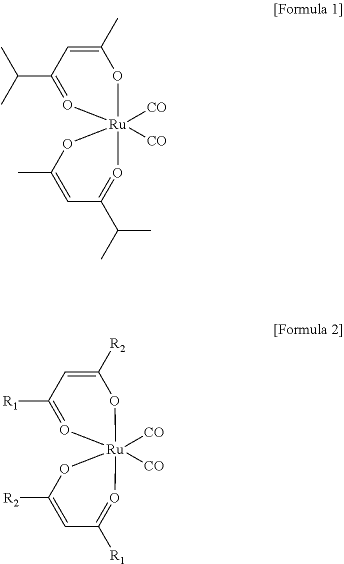

The raw material for chemical deposition containing the organoruthenium compound of the formula 2 has a moderately high vapor pressure, can be in a liquid form at normal temperature, and has favorable basic properties required of a raw material for chemical deposition. Besides, it is applicable to film formation applying hydrogen as a reaction gas, and can inhibit oxidation of a thin film or a substrate with oxygen. When this raw material for chemical deposition is used, a ruthenium thin film having favorable step coverage and a low resistance can be formed. Owing to these many advantages, this raw material for chemical deposition is useful in production process for wirings/electrodes of various semiconductor devices that have been highly integrated and downsized.

### PRIOR ART DOCUMENT

### Patent Document

### Patent Document 1

Japanese Patent Application Laid-Open No. 2003-306472

### Patent Document 2

Japanese Patent No. 4746141

### SUMMARY OF THE INVENTION

### Technical Problem

A conventional raw material for chemical deposition containing an organoruthenium compound described above has a large number of advantages as described above, and can form a high quality ruthenium thin film, and therefore, has entered a stage of practical use. According to further examinations made by the present inventors, however, this organoruthenium compound is found to have a point of improvement for promoting wide use in the future.

This point of improvement is a problem of discoloration or generation of a powder precipitate caused in heating the raw material in a film formation process. In a chemical deposition method, regardless of a specific form thereof, it is necessary to generate a raw material gas by heating/vaporizing a raw material, and to introduce a reaction gas to a reactor (substrate). The organoruthenium compound (Ru complex 1) of the formula 1 described above will be described as an example. The raw material containing this organoruthenium compound is a pale yellow liquid at normal temperature. According to the present inventors, when a heating temperature employed for vaporizing this organoruthenium compound is set to 100° C. or more, and the heating is continued for about 1 month, red discoloration or a precipitate of a red powder is caused in some cases.

In a film formation process by the chemical deposition method, a heating temperature for a raw material is a significant parameter affecting the amount of a raw material gas to be generated. For mass production of semiconductor devices, it is necessary to efficiently produce ruthenium thin films. For this purpose, it is necessary to increase the amount of the raw material to be used as well as to introduce a large amount of the raw material gas onto substrates by increasing the heating temperature so as to increase the vapor pressure of the raw material. Such increase of the heating temperature can be a cause of red discoloration or generation of a red powder.

It is concerned that the discoloration and the red powder thus caused in the raw material may disadvantageously remain as particles in a thin film. Therefore, at a stage before the introduction onto a substrate, the coloration and the powder generation in the raw material should be avoided.

Therefore, an object of the present invention is to reveal a cause of the discoloration and the generation of a powder precipitate caused when a raw material for chemical deposition containing, as a principal component, the organoruthenium compound of the formula 2 including the compound of the formula 1 is heated at a high temperature, and to provide one in which these are inhibited. Besides, with a raw material for chemical deposition containing the organoruthenium compound of the formula 2 applied, a method for forming a stable ruthenium thin film is also provided.

### Solution to Problem

In order to solve the above-described problem, the present inventors have decided to confirm reproducibility of discoloration and generation of a powder precipitate in the organoruthenium compound of the formula 2, and to examine the cause. If the cause of the discoloration and the powder generation in the organoruthenium compound is irreversible like decomposition of the organoruthenium compound, continuous use with generating a raw material gas at a temperature beyond that temperature should be avoided. This is because there is a possibility that the quality of a thin film obtained by the film formation may be degraded, or there is a risk that abrupt thermal decomposition of the raw material compound may be induced. On the other hand, if the cause of the discoloration and the powder generation is not due to decomposition or the like but is a reversible change, there can be a possibility of inhibition thereof while keeping the heating temperature high.

As a result of earnest studies, the present inventors have considered that the cause of the discoloration and the powder generation in the organoruthenium compound of the formula 2 is a reversible change to an intermediate compound through which organoruthenium is synthesized. The details of this reversible change of the organoruthenium compound will now be described in association with a synthesis process of the organoruthenium compound.

The organoruthenium compound of the formula 2 is synthesized, using dodecacarbonyltriruthenium (DCR) as a starting material, by reacting β-diketone with DCR. This synthesis process is represented by the following reaction formula with the organoruthenium compound (Ru complex 1) of the formula 1 used as an example:

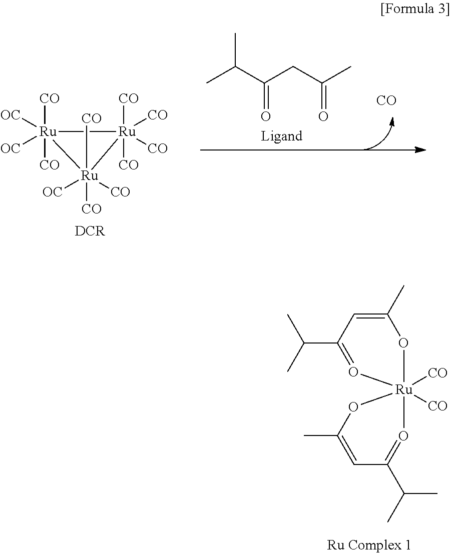

Here, through observation of the appearance in carrying out the synthesis reaction, the present inventors have found that some intermediate compounds are produced in the synthesis reaction before production of the organoruthenium compound. Specifically, in the production process of the organoruthenium compound, DCR and β-diketone are reacted with each other to generate a compound in which one β-diketone (ligand) and two carbonyls are coordinated to one Ru (referred to as the “DCR-ligand adduct”). Then, when the DCR-ligand adduct is formed into a polymer through a polymerization reaction, solubility is lowered to generate a precipitate. Besides, when another β-diketone is coordinated to Ru of the polymer, the polymer is changed into a mononuclear Ru complex 1 having high solubility. It has been revealed that the target compound of the organoruthenium compound is produced through these procedures.

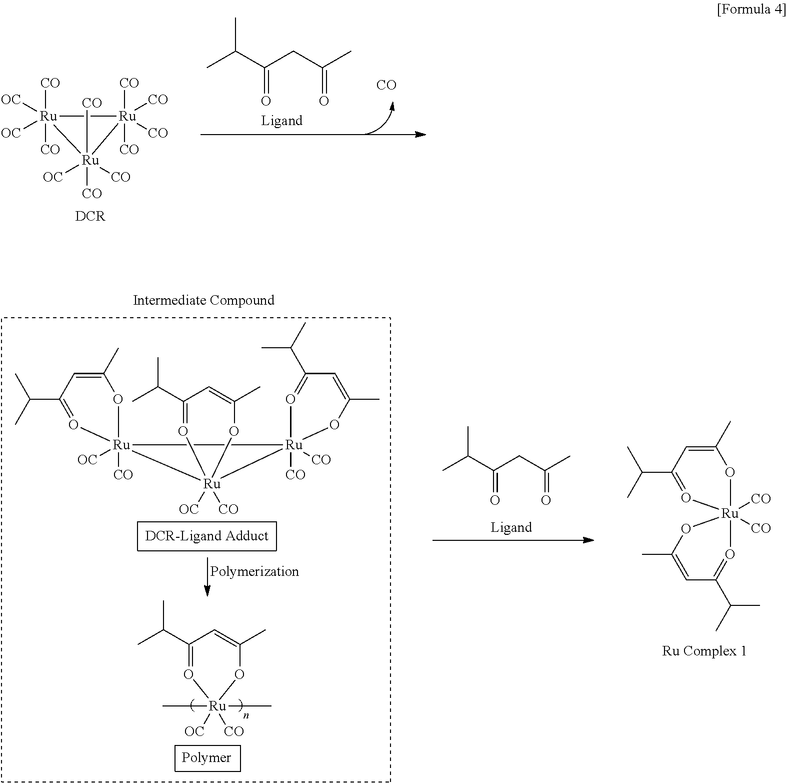

Then, the present inventors have considered that the cause of the discoloration and the red powder generation caused in heating the organoruthenium compound at a high temperature is generation of intermediate compounds such as the DCR-ligand adduct and the polymer described above (which intermediates are hereinafter sometimes referred to as the “polymer and the like”). In other words, the present inventors have considered that when the organoruthenium compound is heated at a high temperature, a part of the organoruthenium compound is returned to the polymer and the like via a reverse reaction, which causes the discoloration and the like.

Besides, the present inventors have presumed that the generation of the polymer and the like via the above-described reverse reaction is a phenomenon different from thermal decomposition. It is presumed that the polymer is generated when one β-diketone is eliminated from the organoruthenium compound at a high temperature. Besides, it is presumed that the DCR-ligand adduct is generated through decomposition of the polymer. Accordingly, it is presumed that when β-diketone is inhibited from eliminating, the polymer is not generated, and the DCR-ligand adduct is not also generated. Thus, it is supposed that stability of the organoruthenium compound is ensured by inhibiting the generation of the polymer and the like. Therefore, the present inventors have made further studies, resulting in finding the following: To a raw material for chemical deposition containing an organoruthenium compound, a β-diketone ligand the same as one contained therein is added, and thus, a raw material for chemical deposition capable of inhibiting generation of the polymer and the like even at a high temperature can be obtained, and in this manner, the present invention has been accomplished.

The present invention that solves the above-described problem is drawn to a raw material for chemical deposition for producing a ruthenium thin film or a ruthenium compound thin film by a chemical deposition method, the raw material for chemical deposition containing an organoruthenium compound represented by the following formula 5, and further containing β-diketone that is represented by the following formula 6, and is the same as a ligand of the organoruthenium compound:

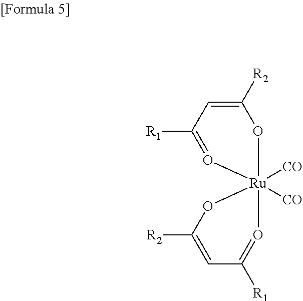

wherein substituents R₁ and R₂ are each hydrogen, or a linear or branched alkyl group.

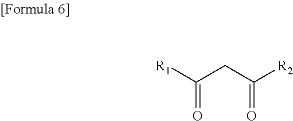

wherein substituents R₁ and R₂ are each hydrogen, or a linear or branched alkyl group.

Now, a raw material for chemical deposition of the present invention and a chemical deposition method of the present invention based on the above-described consideration will be described. It is noted that the raw material for chemical deposition is herein referred to simply as the “raw material” in some cases for simplifying description. It is also noted that “β-diketone that is the same as a ligand of the organoruthenium compound” contained in the raw material of the present invention together with the organoruthenium compound is referred to as the “ligand” in some cases.

### (I) Raw Material for Chemical Deposition of the Invention

As described above, the raw material for chemical deposition of the present invention contains the organoruthenium compound represented by the formula 5, and β-diketone that is the same as the ligand thereof. The respective constitutions will now be described.

### (I-1) Organoruthenium Compound

The organoruthenium compound corresponding to a principal component of the raw material for chemical deposition of the present invention is an organoruthenium compound having the structure of the formula 5, and is a compound in which two β-diketones and two carbonyls are coordinated to ruthenium. The β-diketone has substituents R₁ and R₂, and the substituents R₁ and R₂ are each hydrogen, or a linear or branched alkyl group.

The substituents R₁ and R₂ of the organoruthenium compound are each hydrogen, or a linear or branched alkyl group for making adequate a vapor pressure and a decomposition temperature of the compound, and for ensuring properties priorly required as a raw material for chemical deposition. Both R₁ and R₂ may be hydrogen. Alternatively, at least one of R₁ and R₂ may be a linear or branched alkyl group. When the substituents R₁ and R₂ of the β-diketone are alkyl groups, they are preferably linear or branched alkyl groups having 2 or more and 4 or less carbon atoms. Preferable specific examples of the organoruthenium compound applied in the present invention include the following organoruthenium compounds.

TABLE 1

| | | |
|---|---|---|
| Structural Formula | 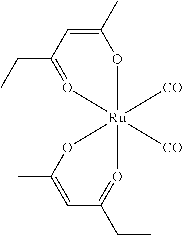 | 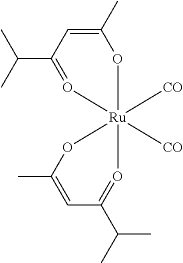 |
| Name | Dicarbonylbis(4-hexen-3-one-5- | Dicarbonylbis(2-methyl-4-hexen-3-one-5- |
| | oxide)ruthenium (II) | oxide)ruthenium (II) (Ru complex 1) |
| Structural Formula | 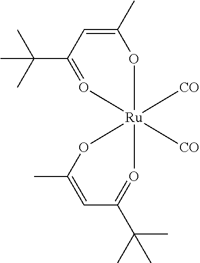 | 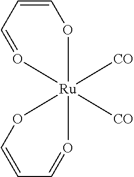 |
| Name | Dicarbonylbis(2,2-dimethyl-4-hexen-3-one- | Dicarbonyl-bis(propen-1-one-3- |
| | 5-oxide)ruthenium (II) | oxide)ruthenium (II) |
| Structural Formula | 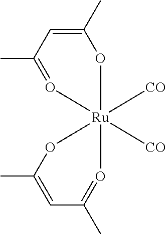 | 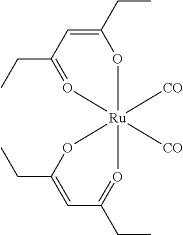 |
| Name | Dicarbonyl-bis(3-penten-2-one-4- | Dicarbonyl-bis(4-hepten-3-one-5- |
| | oxide)ruthenium (II) | oxide)ruthenium (II) |
| Structural Formula | 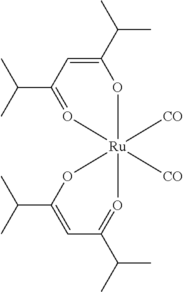 | 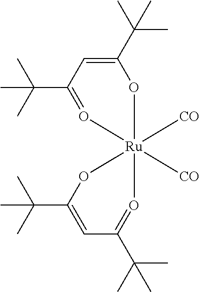 |
| Name | Dicarbonyl-bis(2,6-dimethyl-4-hepten-3-one- | Dicarbonyl-bis(2,2,6,6,-tetramethyl-4- |
| | 5-oxide)ruthenium (II) | hepten-3-one-5-oxide)ruthenium (II) |

It is noted that a ruthenium complex (organoruthenium compound) is formed when an anion generated from β-diketone is coordinated to Ru. At this point, the following anions are generated from β-diketone:

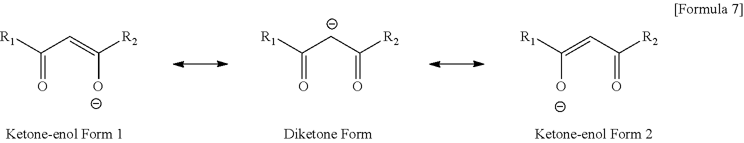

In an actual ruthenium complex, single one of the three anions is rarely coordinated, and in many cases, these anions are coordinated as resonance anions including a plurality of types of anions. Besides, the shape of an anion in a complex is closer to those of the ketone-enol form anions 1 and 2 having a negative charge on oxygen than that of the diketone form anion having a negative charge on carbon. Herein, a structural formula of a ligand of a ruthenium complex is formally indicated using the ketone-enol form anion.

Besides, the ruthenium complex applied in the present invention has a six-coordinated octahedral ligand structure. It is a complex in which two carbonyl groups are coordinated in the cis-form. Therefore, when the substituents R₁ and R₂ of the β-diketone ligand are different, the ruthenium complex can have a structural isomer.

For example, the Ru complex 1 of the formula 1 has the following three structural isomers. Herein, the structural formula of a ruthenium complex is shown without distinguishing such isomers. It is noted, however, that when the complex has isomers, complexes having all the structures are encompassed in the scope of the application of the present invention.

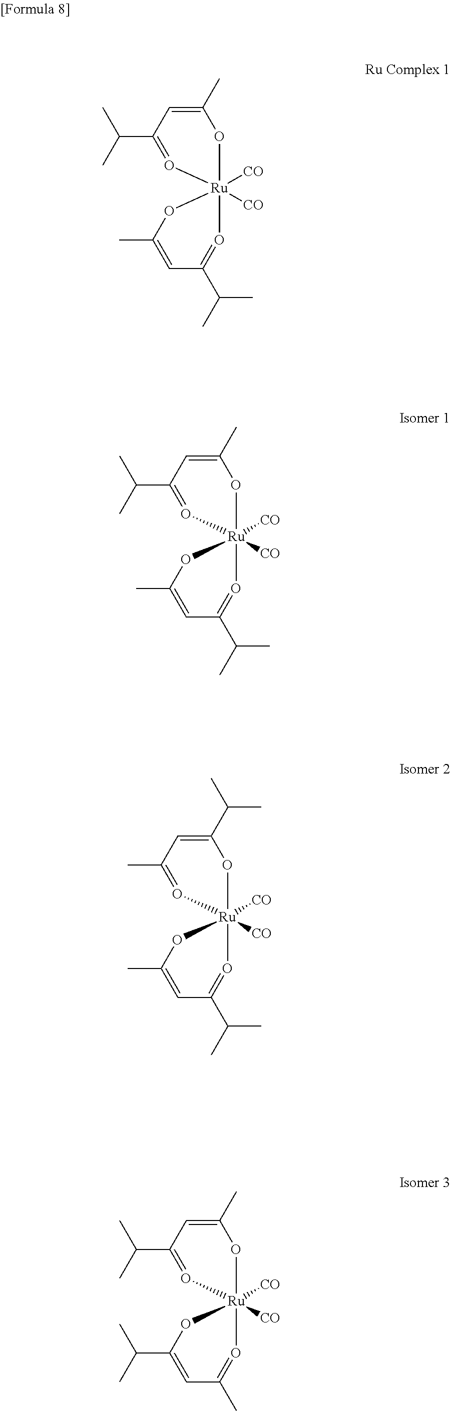

(I-2) Ligand (β-diketone)

The raw material for chemical deposition of the present invention is constituted by adding β-diketone of the formula 6 to the organoruthenium compound of the formula 5. This ligand has substituents R₁ and R₂ that are the same as those of the organoruthenium compound corresponding to the principal component of the raw material for chemical deposition. Preferable ranges thereof are, of course, the same as those of the substituents R₁ and R₂ of the organoruthenium compound described above.

It is noted that the ligand has tautomers such as a diketone form and ketone-enol forms as shown in the following Formula 9. In the ketone-enol forms 1 and 2 of the formula 9, ketone and alcohol are bound to a double bond in the cis-form, and depending on the shape of R₁ and R2, there exists a trans-form ketone-enol form. Herein, the ligand is represented using the structural formula of the diketone form. It is, however, noted that the β-diketone of the present invention is intended to encompass the above-described isomers. Besides, the β-diketone of the present invention encompasses a mixture of these isomers.

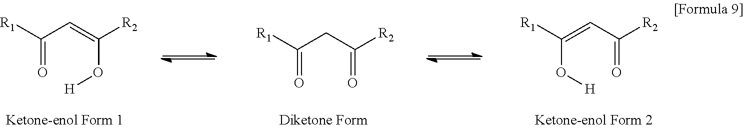

In the raw material for chemical deposition of the present invention, the content of the ligand is preferably 0.3% by mass or more and 10% by mass or less with respect to the mass of the organoruthenium compound. When the content is less than 0.3% by mass, it is difficult to inhibit the generation of the polymer and the like at a high temperature, and hence the discoloration and the powder precipitate are caused in the raw material. On the other hand, when the content exceeds 10% by mass, the effect of inhibiting the generation of the polymer and the like does not differ. Besides, when the ligand is added in an excessive amount, it is concerned that the physical properties/vaporization properties of the entire raw material may be changed to affect the film formation process. The content of the ligand is more preferably 0.4% by mass or more and 5% by mass or less, and particularly preferably 0.5% by mass or more and 5% by mass or less.

The content of the ligand in the raw material for chemical deposition can be quantitatively determined through composition analysis by NMR or the like. When the raw material for chemical deposition of the present invention is subjected to NMR analysis, a signal derived from the added ligand appears. Based on the integral ratio of the signal, the molar composition ratio and the mass ratio of the ligand can be calculated.

### (II) Chemical Deposition Method for Ruthenium Thin Film of the Invention

Regarding the present invention described so far, the idea that β-ketone having the same ligand is added to the ruthenium compound used as a raw material is useful also for a chemical deposition method for a ruthenium thin film and a ruthenium compound thin film. A chemical deposition method of the present invention is the same, in the basic process, as a general method. In a chemical deposition method, a raw material for chemical deposition is heated to obtain a material gas, and the material gas is heated to a prescribed film forming temperature while being introduced onto a substrate surface. Thus, decomposition of the organoruthenium compound and precipitation of ruthenium are caused on the substrate surface, resulting in forming a ruthenium thin film or a ruthenium compound thin film. The present invention also follows this basic process. Based on the features of the present invention described so far, however, the chemical deposition method of the present invention is roughly divided into the following three patterns in accordance with an aspect of the addition of a ligand to a raw material for chemical deposition (raw material gas).

### (II-1) First Chemical Deposition Method of the Invention

This chemical deposition method is a chemical deposition method characterized by using, in the chemical deposition method for a ruthenium thin film or a ruthenium compound thin film described above, the raw material for chemical deposition of the present invention as the raw material for chemical deposition. In the first chemical deposition method, the raw material for chemical deposition of the present invention to which the ligand is precedently added is used to be heated to a desired temperature, and hence the raw material gas can be rapidly generated without causing the discoloration and the powder generation.

The first chemical deposition method is characterized only by the use of the raw material for chemical deposition of the present invention as a raw material, and procedures following the generation of the raw material gas by heating the raw material are the same as those of the conventional chemical deposition method. In the procedure for heating the raw material, the organoruthenium compound of the present invention can be directly heated, or a solution obtained by dissolving it in an appropriate solvent may be heated. A heating temperature for the raw material in this procedure is preferably 0° C. or more and 300° C. or less. In this chemical deposition method, thermal stability of the organoruthenium compound has been improved owing to the ligand contained therein, and there is a low possibility of generation of the polymer and the like, and therefore, the heating can be performed at a high temperature of 200° C. or more.

Besides, the heating of the raw material can be performed a plurality of times before introducing the raw material gas into a reactor. For example, the raw material can be heated in two stages of first heating at a comparative low temperature (of 150° C. or less) for vaporization, and then of heating at a high temperature.

The raw material gas is joined with an appropriate carrier gas to be transported onto a substrate. As the carrier gas, an inert gas (such as argon or nitrogen) is preferably used. For efficiently forming a ruthenium thin film, a reaction gas is preferably introduced together with the raw material gas. As the reaction gas, a reducing gas of hydrogen or the like can be used. As the reaction gas, reducing gas species such as ammonia, hydrazine, and formic acid can be applied in addition to hydrogen, and from the viewpoint of preventing oxidation of a ruthenium thin film and a substrate, such a reaction gas is preferably applied. The organoruthenium compound applied in the present invention can be, however, decomposed even when oxygen is used as the reaction gas. Therefore, unless application of an oxygen gas is avoided, oxygen can be applied as the reaction gas. Such a reaction gas can work also as a carrier gas, and hence, the application of the above-described carrier gas containing an inert gas or the like is not essential.

Then, the raw material gas is transported to a reactor together with a carrier gas and an appropriate reaction gas, and is heated on a substrate surface to form a ruthenium thin film. At this point, as film formation conditions, conditions set for a conventional ruthenium compound of the formula 2 can be applied. This is because the raw material for chemical deposition of the present invention is not different in the vaporization properties/decomposition properties of the organoruthenium compound.

A film forming temperature employed in the film formation is preferably 100° C. or more and 400° C. or less. When the temperature is less than 100° C., the decomposition reaction of the organoruthenium compound is difficult to proceed, and hence the film cannot be efficiently formed. On the other hand, when the film forming temperature is as high as over 400° C., the film is difficult to be uniformly formed, and it is disadvantageously concerned that the substrate may be damaged, or the like. It is noted that the film forming temperature is usually adjusted by a heating temperature of the substrate.

### (II-2) Second Chemical Deposition Method of the Invention

The second chemical deposition method of the present invention is a method in which a raw material containing only an organoruthenium compound is used as in the conventional method but a ligand is added to the raw material before generation or during generation of a raw material gas. In other words, this method is a chemical deposition method characterized by using an organoruthenium compound represented by the following formula 10 as a raw material for chemical deposition, and adding, before heating or during heating the raw material for chemical deposition, β-diketone that is represented by the following formula 11, and is the same as a ligand of the organoruthenium compound.

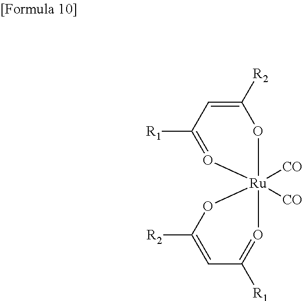

wherein substituents R₁ and R₂ are each hydrogen, or a linear or branched alkyl group.

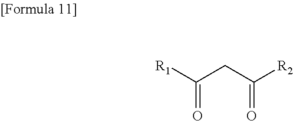

wherein substituents R₁ and R₂ are each hydrogen, or a linear or branched alkyl group.

Even when a raw material for chemical deposition containing only the organoruthenium compound is used as in this second chemical deposition method, the generation of the polymer and the like can be inhibited by adding the ligand before the heating or during the heating for generating the raw material gas. It is noted that the raw material containing only the organoruthenium compound of the formula 10 means that an organic compound that can possibly directly affect a reaction for forming a ruthenium thin film (decomposition reaction of the organoruthenium compound/precipitation reaction of ruthenium) is not contained. In other words, it is intended that use of a solvent or an additive not directly contributing to the film forming reaction is not excluded. Therefore, the organoruthenium compound of the formula 10 may be directly heated, or a solution obtained by dissolving it in an appropriate solvent may be heated.

As a method for adding the ligand to the raw material for chemical deposition containing only the organoruthenium compound, the ligand may be directly added to a raw material container, or the raw material container may be provided with a pipe for adding the ligand so as to add the ligand through the pipe. Besides, the amount of the ligand added is preferably 0.3% by mass or more and 10% by mass or less with respect to the mass of the organoruthenium compound, more preferably 0.4% by mass or more and 5% by mass or less, and particularly preferably 0.5% by mass or more and 5% by mass or less.

The film forming method and conditions to be employed after adding the ligand to the raw material can be the same as those employed in the first chemical deposition method described above. The heating temperature of the raw material, the conditions related to a reaction gas/carrier gas, and further the film forming temperature can be also the same as those employed in the first chemical deposition method.

### (II-3) Optional Operation in First and Second Chemical Deposition Methods

In the chemical deposition method, the raw material is continuously heated during the film formation. At this point, in the first and second chemical deposition methods in which the ligand is contained in the raw material, the content of the ligand in the raw material may vary in accordance with the progress of the film formation in some cases. For example, there is a possibility that the ligand may be priorly vaporized to be reduced as compared with the initial content depending on a difference in the vaporization properties between the organoruthenium compound and the ligand, and the heating/bubbling conditions and the like. Therefore, there is a possibility that the polymer and the like may be generated in the raw material due to the reduction of the content of the ligand. Alternatively, the content of the ligand may be increased on the contrary, and in this case, it is concerned that the vaporization properties of the entire raw material may be affected. Therefore, in the first and second chemical deposition methods, it is preferable, in some cases, that the content of the ligand in the raw material is retained within a range of 0.3% by mass or more and 10% by mass or less with respect to the mass of the organoruthenium compound. The range is more preferably 0.4% by mass or more and 5% by mass or less, and particularly preferably 0.5% by mass or more and 5% by mass or less.

As a method for retaining the content of the ligand in the raw material, addition of the ligand to the raw material during the film formation can be employed. Similarly, the organoruthenium compound can be added to the raw material. Specifically, a small amount of the ligand can be added at regular time intervals for adjusting the content of the ligand in the raw material. At this point, the amount of the ligand added and the timing of the addition can be adjusted in accordance with the heating conditions for the raw material and the film formation conditions.

Besides, the variation in the content of the ligand in the raw material can affect the content of the ligand in the raw material gas. Therefore, a mixing ratio of the ligand contained in the raw material gas may be retained in a prescribed range. In this case, the mixing ratio of the ligand in the raw material gas is retained preferably in a range of 0.9% or more and 30% or less in terms of a molar ratio with respect to the organoruthenium compound. The range is more preferably 1.2% or more and 15% or less, and particularly preferably 1.5% or more and 15% or less in terms of a molar ratio.

The method for adding the ligand to the raw material gas is not especially limited. For example, a pipe for a gas containing the ligand may be joined to a pipe for the raw material, or the raw material gas and the ligand may be held to be mixed in an appropriate one of vessels and towers. For the addition of the ligand, only the ligand may be added to the raw material gas, or the ligand may be mixed with a carrier gas or a reaction gas so as to add the resultant to the raw material gas. It is noted that this operation for adding the ligand to the raw material gas may be performed together with the addition of the ligand to the raw material described above, or may be singly executed.

The operations related to the content of the ligand in the raw material and the raw material gas described above have an effect of inhibiting the generation of the polymer and the like by compensating the content decrement of the ligand in the raw material. Besides, it also has an effect that the content can be adjusted in accordance with the heating temperature of the raw material. It is, however, noted that these operations are not essential but optional.

### Advantageous Effects of the Invention

As described so far, in the present invention, it has been found that the cause of discoloration and generation of a powder precipitate at a high temperature in the raw material for chemical deposition containing an organoruthenium compound of the formula 2 as a principal component is generation of intermediate compounds such as a polymer and the like through reverse reactions. The present invention reveals that a ligand (3-diketone) of an organoruthenium compound is added to the organoruthenium compound as effective means for inhibiting the generation of the polymer and the like.

A raw material for chemical deposition and a chemical deposition method of the present invention can produce a ruthenium thin film and a ruthenium compound thin film more stably than in conventional technique with retaining favorable properties of the organoruthenium compound of the formula 2. In particular, mass production of a raw material gas by employing a high temperature can be made possible.

### BRIEF DESCRIPTION OF THE DRAWINGS

FIGS. 1(a) and 1(b) are a diagram of infrared adsorption spectra of a red powder collected from an organoruthenium compound after a heating test described in First Embodiment, and a synthesized red powder (polymer);

FIGS. 2(a) and 2(b) are a diagram of NMR spectra of an organoruthenium compound in which a ligand is added in First Embodiment;

FIG. 3 illustrates photographs of results of heating an organoruthenium compound at 140° C., 170° C., and 200° C. for 80 hours;

FIG. 4 illustrates photographs of results of a heating test (140° C. or 170° C., 7 days) performed with 3-diketone, CO, BHT added to organoruthenium compounds; and

FIG. 5 illustrates photographs of results of a heating test (200° C., 7 days) performed with 0.1° A by mass, 0.25% by mass, 0.5% by mass, and 1° A by mass of a ligand added to organoruthenium compounds.

### DETAILED DESCRIPTION OF THE PREFERRED EMBODIMENTS

First Embodiment: An embodiment of the present invention will now be described. In the present embodiment, an initial heating test for checking whether or not discoloration or generation of a powder precipitate was caused in heating an organoruthenium compound of the formula 2 was carried out, and then, a test for checking a chemical structure of the powder precipitate was carried out. Besides, a heating test for checking an effect of inhibiting the discoloration and the like by addition of a ligand to the organoruthenium compound was carried out.

### [Initial Heating Test]

As the organoruthenium compound, a Ru complex 1 of the formula 1 (product name: Carish, manufactured by TANAKA Kikinzoku Kogyo, K.K.) was prepared. This organoruthenium compound is a product obtained through a reaction between a starting material of dodecacarbonyltriruthenium (DCR) and 5-methylhexane-2,4-dione, and is a yellow transparent liquid at room temperature. In an inert gas atmosphere, 4.00 g of the organoruthenium compound was weighed out, and enclosed in a closed glass container. The glass container was set in a heating oven to be heated at 140° C., 170° C., or 200° C. for 80 hours. After heating for 80 hours, each sample was taken out of the heating oven, and the appearance of the organoruthenium compound in the container was observed to check whether or not discoloration and a powder precipitate had been caused.

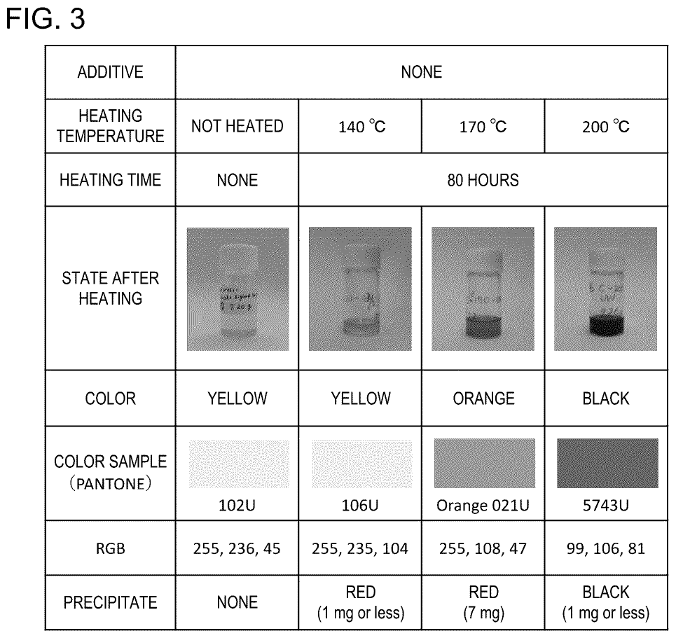

Results of this heating test are illustrated in FIG. 3. The sample heated at 140° C. was a yellow transparent liquid, and was in substantially the same state as that before the heating. On the other hand, the sample heated at 170° C. had changed to an orange liquid, and thus, discoloration was confirmed. Besides, a red powder precipitate was generated on the bottom of the container. The sample heated at 200° C. was changed in color to black, and a small amount of a black deposition was generated therein. Examining these results of the heating test, it is presumed that the change caused in the heating at 200° C. is due to thermal decomposition of the organoruthenium compound. It was thus confirmed that the discoloration and the powder precipitate in the organoruthenium compound, that is, the problem of the present invention, are caused in heating at 170° C.

### [Synthesis of Polymer, and Test for Checking Chemical Structure of Red Precipitate]

In order to examine the composition of the red powder generated in the sample heated at 170° C. in the heating test described above, a polymer of the organoruthenium compound was synthesized for comparison. The organoruthenium compound (formula 2) corresponding to the target of the present invention is synthesized by reacting 2 equivalents of the ligand (β-diketone) with Ru contained in DCR. Therefore, it is presumed that a polymer can be synthesized by reacting 1 equivalent of β-diketone with Ru of DCR for making the synthesis of the organoruthenium compound incomplete.

In this consideration, a polymer was synthesized. 5.00 g of dodecacarbonyltriruthenium (DCR) (manufactured by TANAKA Kikinzoku Kogyo K.K., 7.82 mmol), and 3.01 g of 5-methylhexane-2,4-dione (manufactured by TANAKA Kikinzoku Kogyo K.K., 23.46 mmol) that were raw materials of the polymer were added to 100 mL of dry decane. The resultant was reacted by heating in an oil bath at 160° C. for 20 hours in a nitrogen gas atmosphere. Thereafter, the resultant was cooled to room temperature. As a result of this synthesis operation, 3.16 g of a synthesized product in the form of a red powder was obtained. This red powder had very low solubility in a general solvent, and hence could not be subjected to NMR measurement. Therefore, for assignment of the compound, elemental analysis and measurement of an infrared adsorption spectrum were performed.

Next, the red powder obtained by heating the organoruthenium compound at 170° C. in the above-described heating test was filtered out from the sample to collect about 7 mg of the red powder. The collected red powder was subjected to elemental analysis and measurement of an infrared adsorption spectrum.

Results of the elemental analysis of the red powder obtained by the above-described heating test, and the red powder (polymer) obtained by the synthesis are shown in Table 2. Table 2 also shows theoretical values obtained by calculating constituent elements of the polymer and content ratios thereof based on the molecular structure.

TABLE 2

| | C/% | H/% | N/% |
|---|---|---|---|
| Red precipitate generated in heating test | 37.89 | 4.01 | 0.00 |
| Synthesized red precipitate | 37.77 | 3.89 | 0.00 |
| Theoretical value of polymer | 38.03 | 3.90 | 0.00 |

It was found from Table 2 that content ratios of carbon, hydrogen and nitrogen in the red powder obtained by the heating test and the red powder obtained by the synthesis sufficiently accord with theoretical values of carbon, hydrogen, and nitrogen calculated based on the molecular structure of the polymer (with an error within 0.30%).

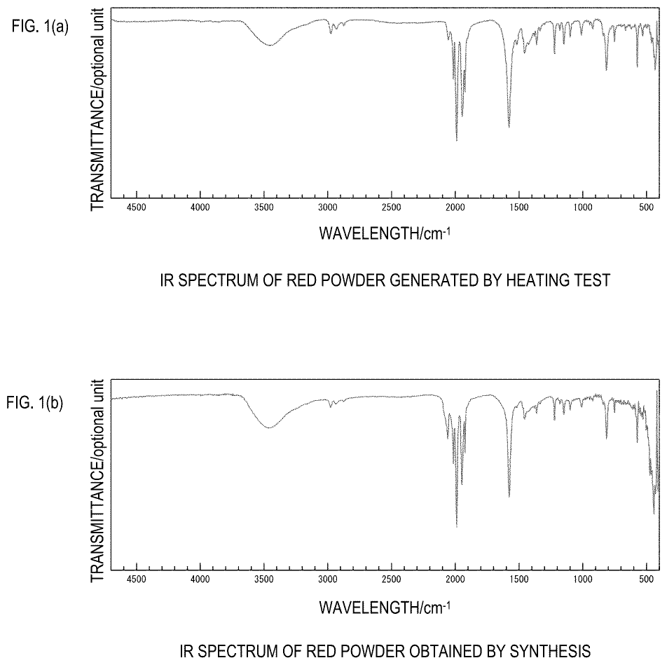

Besides, FIGS. 1(a) and 1(b) illustrate an infrared adsorption spectrum of the red powder obtained by the heating test and an infrared adsorption spectrum of the red powder obtained by the synthesis. As is understood from FIGS. 1(a) and 1(b), adsorption peaks were observed in the same wavelength regions in both the spectra, and hence it can be determined that these powders are the same substance.

Based on the results of the elemental analysis and the results of the measurement of the infrared adsorption spectra, it is supposed that there is a high possibility that a red powder generated by heating an organoruthenium compound at a high temperature is a polymer corresponding to an intermediate compound generated in the synthesis process of the organoruthenium compound. Besides, it is supposed that the discoloration of the organoruthenium compound is also caused similarly by the generation of the polymer. Regarding the theoretical values of the elemental analysis estimated based on the molecular structure, however, the polymer and the DCR-ligand adduct have the same values, and therefore, it is not decided here that the cause of the discoloration and the red powder caused by high-temperature heating of the organoruthenium compound is the generation of the polymer alone. A possibility that the DCR-ligand adduct is generated together with the generation of the polymer, or instead of the polymer cannot be denied. In any case, the followings were confirmed based on the results of these confirmation tests:
(1) When the organoruthenium compound (formula 2) is heated at a high temperature, the substance (red precipitate) having the same theoretical values as the “polymer” is generated. This substance is different from the organoruthenium compound and DCR.
(2) The red precipitate generated in the heating test and the synthesized red precipitate both have the same elemental analysis values as the “polymer”.
   [Confirmation Test for Effect of Inhibiting Generation of Polymer and the like by Adding Ligand]

It was confirmed, based on the results of these preliminary tests, that when the organoruthenium compound of the formula 2 is heated at a high temperature (170° C.), the discoloration and the powder precipitate are generated, and that there is a high possibility that the cause is the generation of the polymer. Therefore, the effect of inhibiting the generation of the polymer by adding a ligand was checked.

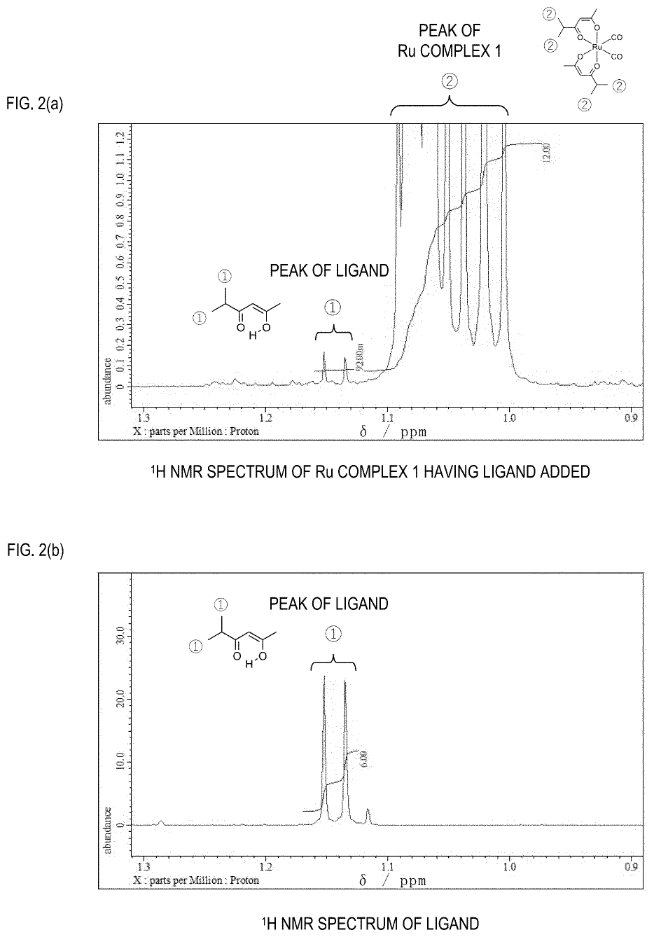

4.00 g (9.72×10⁻³ mol) of the organoruthenium compound (Ru complex 1) the same as that used in the initial heating test was weighed out, and with respect to the mass of the organoruthenium compound, 0.48% by mass (0.019 g) of 5-methylhexane-2,4-dione as the ligand was added thereto to be mixed. The 1 H NMR spectrum of the organoruthenium compound having the ligand added thereto is illustrated in FIGS. 2(a) and 2(b). As is understood from FIGS. 2(a) and 2(b), a peak derived from the ligand was clearly observed in the organoruthenium compound having the ligand added thereto. It was thus confirmed that In the raw material for chemical deposition of the present invention, even when the amount of the ligand added is less than 1% by mass, the presence can be easily discriminated. It is noted that the content of the ligand can be measured by calculating a molar ratio between the organoruthenium compound and the ligand based on peak areas of NMR.

Next, the organoruthenium compound to which 1% by mass of the ligand had been added was subjected to a heating test. The heating test was carried out under the same conditions as those for the initial heating test with the heating temperature set to 140° C. or 170° C. and with the heating time set to 7 days.

Besides, in this heating test, an organoruthenium compound having another additive added thereto was also subjected to the heating test for comparison with the effect of the addition of the ligand. As a sample using another additive, a sample obtained by bubbling an organoruthenium compound with carbon monoxide (amount of CO added: about 1% by mass) was produced. This is for checking the effect of adding a carbonyl ligand (CO), that is, the other ligand of the organoruthenium compound of the formula 2. Besides, a sample obtained by adding dibutylhydroxytoluene (BHT), that is, an antioxidant, to an organoruthenium compound in an amount of 1% by mass with respect to the organoruthenium compound was also produced. The samples respectively having CO and BHT added thereto were subjected to a heating test at 140° C. for 7 days.

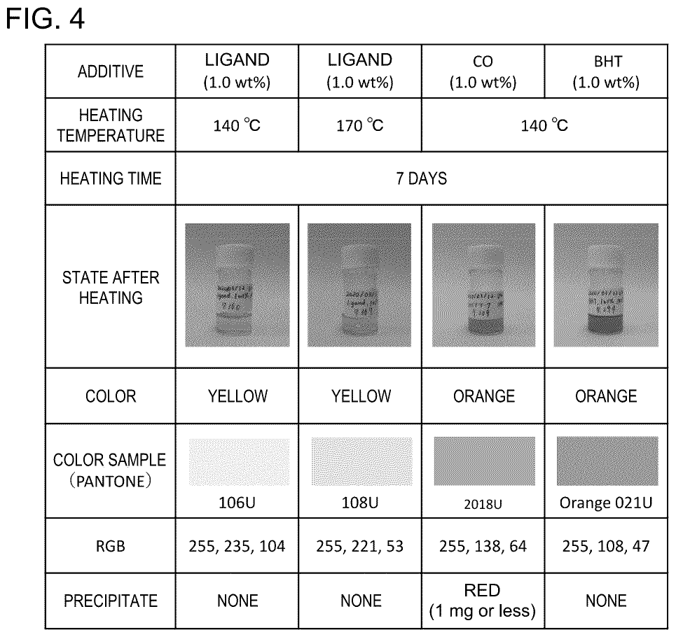

FIG. 4 illustrates photographs of results of this heating test. The organoruthenium compound having the ligand added thereto retained the yellow transparent state before the heating test even after heated at either temperature. In neither of the respective samples after the heating test, discoloration to red/orange was observed, nor a red powder precipitate was observed. On the other hand, the sample having CO added thereto and the sample having BHT added thereto were changed in color to orange when heated at 140° C.

Accordingly, for inhibition of the discoloration and the powder precipitate caused in the organoruthenium compound by heating, it is effective to add β-diketone having the same structure as the ligand to the organoruthenium compound. It is deemed that this effect is an effect that cannot be obtained by addition of the other ligand (carbonyl ligand) or a general degradation inhibitor (antioxidant). It was confirmed based on the various tests of First Embodiment described so far that the raw material for chemical deposition containing the organoruthenium compound having the ligand added thereto of the present invention has the effect of inhibiting discoloration and powder generation due to generation of the polymer and the like otherwise caused when heated at a high temperature.

### Second Embodiment

In the present embodiment, a heating test for confirming the effect depending on the amount of the ligand added was carried out. Samples were prepared by respectively adding 0.1% by mass, 0.25% by mass, 0.3% by mass, 0.5% by mass, and 1% by mass of the ligand (5-methylhexane-2,4-dione) to the same organoruthenium compound (Ru complex 1) (product name: Carish) as that used in First Embodiment.

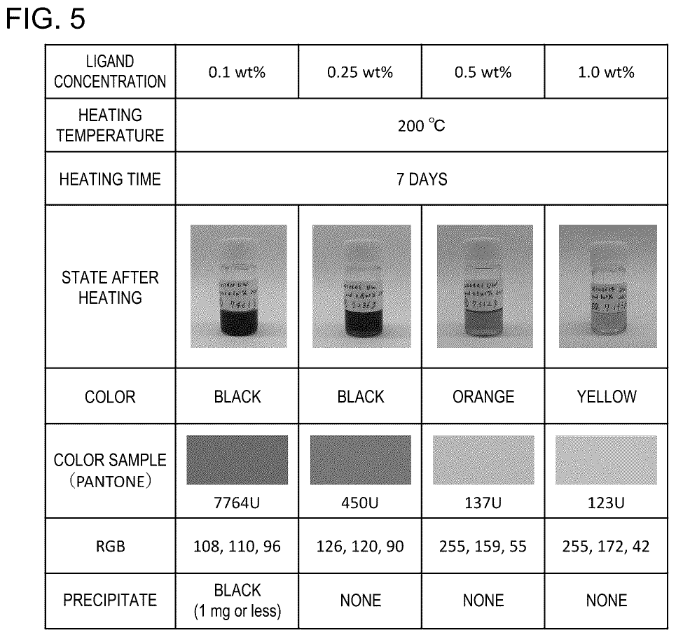

Each of the thus prepared samples was subjected to a heating test at 200° C. for 7 days. Results of this heating test are illustrated in FIG. 5. As a result, the organoruthenium compounds in which 0.1% by mass or 0.25% by mass of the ligand was added were changed in color to black. Besides, in the sample in which the amount of the ligand added was 0.1% by mass, a small amount (1 mg or less) of a black precipitate was generated. On the other hand, the organoruthenium compounds in which 0.5% by mass or 1.0% by mass of the ligand was added were little changed in color and the color was yellow to orange, and no precipitate was generated. Although not illustrated in FIG. 5, the same results were obtained when the amount was 0.3% by mass. Accordingly, it was confirmed that an organoruthenium compound having 0.3% by mass or more of the ligand added thereto was highly stable against heat.

### Third Embodiment

In the present embodiment, a film formation test for a ruthenium thin film using a raw material for chemical deposition containing an organoruthenium compound containing a ligand was carried out.

A raw material for chemical deposition was prepared by adding, to the same organoruthenium compound (Ru complex 1) as that used in First Embodiment, 1.0% by mass of the ligand of 5-methylhexane-2,4-dione with respect to the mass of the organoruthenium compound. This raw material for chemical deposition was used for forming a ruthenium thin film with a CVD apparatus. Film formation conditions were as follows:
Substrate: Si, or SiO₂
Raw Material Heating Temperature: 180° C.
Carrier Gas: nitrogen (50 sccm)
Reaction Gas: hydrogen (500 sccm)
Pressure: 4000 Pa
Substrate Temperature: 350° C.
Film Forming Time: 60 min

As a result of this film formation test, a ruthenium thin film with a thickness of 30 nm could be formed on both Si and SiO₂ substrates. No particles were observed on the thin film, and the ruthenium thin film was confirmed to have a smooth surface. Besides, the raw material (organoruthenium compound) after the film formation test was not changed in color, and thus, it was confirmed that the effect of inhibiting thermal decomposition in heating the raw material is thus obtained.

### INDUSTRIAL APPLICABILITY

According to a raw material for chemical deposition and a chemical deposition method of the present invention, when a raw material for chemical deposition principally containing prescribed organoruthenium compound is applied, discoloration/powder generation in the organoruthenium compound can be inhibited even when a heating temperature of a raw material is set to a high temperature. In this case, properties of the organoruthenium compound can be retained, and a ruthenium thin film and a ruthenium compound thin film can be produced more stably than in conventional technique. The present invention is useful for production of wirings/electrodes of various semiconductor devices by a chemical deposition method (CVD or ALD), and in particular, is applicable to mass production of these.

1. A raw material for chemical deposition for producing a ruthenium thin film or a ruthenium compound thin film by a chemical deposition method, the raw material for chemical deposition comprising:
an organoruthenium compound represented by the following formula 1, and further comprising:
β-diketone that is represented by the following formula 2, and is the same as a ligand of the organoruthenium compound: 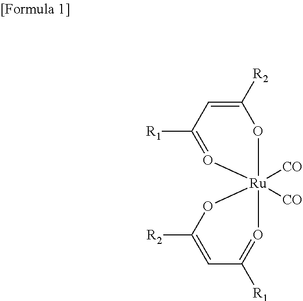
wherein substituents R₁ and R₂ are each hydrogen, or a linear or branched alkyl group; 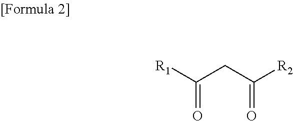
wherein sub stituents R₁ and R₂ are each hydrogen, or a linear or branched alkyl group.

2. The raw material for chemical deposition according to claim 1, wherein a content of the ligand is 0.3% by mass or more and 10% by mass or less with respect to a mass of the organoruthenium compound.

3. A chemical deposition method for a ruthenium thin film or a ruthenium compound thin film, comprising heating a raw material for chemical deposition to obtain a raw material gas, and heating the raw material gas while introducing to a substrate surface,
wherein the raw material for chemical deposition defined in claim 1 or 2 is used as the raw material for chemical deposition.

4. A chemical deposition method for a ruthenium thin film or a ruthenium compound thin film, comprising heating a raw material for chemical deposition to obtain a raw material gas, and heating the raw material gas while introducing to a substrate surface,
wherein the method comprises:
using a raw material for chemical deposition containing an organoruthenium compound represented by the following formula 3 as the raw material for chemical deposition; and
adding, before or during the heating of the raw material for chemical deposition, β-diketone that is represented by the following formula 4 and is the same as a ligand of the organoruthenium compound, 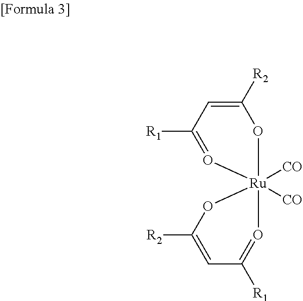
wherein substituents R₁ and R₂ are each hydrogen, or a linear or branched alkyl group; and 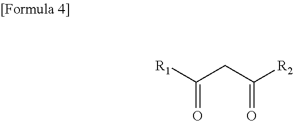
wherein substituents R₁ and R₂ are each hydrogen, or a linear or branched alkyl group.

5. The chemical deposition method according to claim 4, wherein an amount of the ligand added is 0.3% by mass or more and 10% by mass or less with respect to a mass of the organoruthenium compound.

6. The chemical deposition method according to claim 3, wherein a content of the ligand contained in the raw material for chemical deposition is retained at 0.3% by mass or more and 10% by mass or less with respect to a mass of the organoruthenium compound.

7. The chemical deposition method according to claim 3, wherein a mixing ratio of the ligand contained in the raw material gas is retained at 0.9% or more and 30% or less in terms of a molar ratio with respect to the organoruthenium compound.

8. A chemical deposition method for a ruthenium thin film or a ruthenium compound thin film, comprising heating a raw material for chemical deposition to obtain a raw material gas, and heating the raw material gas while introducing to a substrate surface,
wherein the raw material for chemical deposition defined in claim 2 is used as the raw material for chemical deposition.

9. The chemical deposition method according to claim 4, wherein a content of the ligand contained in the raw material for chemical deposition is retained at 0.3% by mass or more and 10% by mass or less with respect to a mass of the organoruthenium compound.

10. The chemical deposition method according to claim 5, wherein a content of the ligand contained in the raw material for chemical deposition is retained at 0.3% by mass or more and 10% by mass or less with respect to a mass of the organoruthenium compound.

11. The chemical deposition method according to claim 4, wherein a mixing ratio of the ligand contained in the raw material gas is retained at 0.9% or more and 30% or less in terms of a molar ratio with respect to the organoruthenium compound.

12. The chemical deposition method according to claim 5, wherein a mixing ratio of the ligand contained in the raw material gas is retained at 0.9% or more and 30% or less in terms of a molar ratio with respect to the organoruthenium compound.
